(19) United States
(12) Patent Application Publication
(10) Pub. No.: US 2026/0283508 A1
(43) Pub. Date: Sep. 24, 2026

(54) MEASUREMENT OF BETA BAND VIBRATIONS SIGNAL IN A SUBJECT

(71) Applicant: UNIVERSITÉ LIBRE DE BRUXELLES, Bruxelles (BE)

(72) Inventor: Mathieu BOURGUIGNON, Uccle (BE)

(21) Appl. No.: 19/476,071

(22) PCT Filed: Apr. 17, 2024
(86) PCT No.: PCT/EP2024/060326

§ 371 Date: Oct. 16, 2025

(30) Foreign Application Priority Data
Apr. 20, 2023 (EP) 23168860.7

(51) Int. Cl. A61B 5/11; A61B 5/00

(52) U.S. Cl. CPC A61B 5/1124; A61B 5/1107; A61B 5/4064; A61B 5/4082; A61B 5/4842; A61B 5/742

(57) ABSTRACT

Provided is a system or computer-implemented method for determining a proxy for cerebral cortex-detected beta sensorimotor cortical rhythms of a subject, a proxy signal wherein the proxy signal is correlated to the cerebral cortex-detected beta sensorimotor cortical rhythms of the subject, the method comprising:
receiving a dataset containing measurements from a vibration sensing unit configured to measure mechanical vibrations of a body part, the measurements previously acquired from the subject during a measurement period and during a sustained contraction effort,
extracting from the dataset a beta-band vibration signal, a BBV signal, which is signal representative of a temporal envelope of the measured mechanical vibrations filtered within a beta frequency band,
   wherein the proxy signal is the BBV signal, or is derived from the BBV signal.

### CROSS REFERENCE TO RELATED APPLICATIONS

The present application is a U.S. National Phase of International Application No. PCT/EP2024/060326 entitled “MEASUREMENT OF BETA BAND VIBRATIONS SIGNAL IN A SUBJECT,” and filed on Apr. 17, 2024. International Application No. PCT/EP2024/060326 claims priority to European Patent Application No. 23168860.7 filed on Apr. 20, 2023. The entire contents of each of the above-listed applications are hereby incorporated by reference for all purposes.

### FIELD OF THE INVENTION

The present invention is in a field of beta sensorimotor cortical rhythm measurements. In particular, it is in a field of a replacement electroencephalogram (EEG) and/or magnetoencephalogram (MEG) technique for measurement of these signals, and for a reduced-cost and -complexity measurement of neurological health.

### BACKGROUND

The sensorimotor areas of the brain control the movements and integrate sensory information to guide them. In humans, these areas display rhythms in the brainwave beta (13-40 Hz) and sometimes brainwave alpha (8-13 Hz) frequency bands, here collectively referred to as beta rhythms, which are reflective of their state of activation. Beta rhythms undergo modulations in response to movements and many types of stimulations, making them a particularly abundant subject of fundamental and translational research, and a marker for neurological health.

Measuring EEG and/or MEG beta sensorimotor cortical rhythms in the brain of the subject require specialised equipment and expertise of a specialist to carry out. This creates a barrier to measuring in remote locations, economically deprived areas, and for regular monitoring.

The present invention aims to overcome the problems of the art.

### SUMMARY

Provided herein is a computer-implemented method for determining a proxy for cerebral cortex-detected beta sensorimotor cortical rhythms of the subject, proxy signal (122), wherein the proxy signal is correlated to the cerebral cortex-detected beta sensorimotor cortical rhythms of the subject, the method comprising:
receiving a dataset containing measurements from a vibration sensing unit (200) configured to measure mechanical vibrations of a body part (300), the measurements previously acquired from the subject during a measurement period and during a sustained contraction effort;
extracting from the dataset a beta-band vibration signal, BBV signal (120), which is a signal representative of a temporal envelope of the measured mechanical vibrations filtered within a beta frequency band;
wherein the proxy signal (122) is the BBV signal (120), or is derived from the BBV signal (120).

Provided herein is a computer-implemented method for determining a beta band vibrations, BBV, signal (120) of a subject, the method comprising:
receiving a dataset containing measurements from a vibration sensing unit configured to measure mechanical vibrations of a body part (300) during a sustained contraction effort of the subject, the measurements acquired during a measurement period and during the sustained contraction effort,
extracting from the dataset a beta-band vibration signal, BBV signal (120), which is a signal representative of an amplitude of the measured mechanical vibrations within a beta frequency band.

The measurement period may contain a first sub-period and a second sub-period of measurement,
during the first sub-period of measurement, the sustained contraction effort is at a constant intensity level,
during the second sub-period of measurement, the subject is engaged in a task.

The task, Task T1, may be physical movement performed by the subject and the subject maintains the sustained contraction effort at the constant intensity level, or
the task, Task T2, may be change in intensity of the sustained contraction effort by the subject from the constant, first, intensity level of the first sub-period to a second intensity level different from the first intensity level,
the task, Task T3, may be visual observing of or an imagining of a physical movement by the subject and the subject maintains the sustained contraction effort at the constant intensity level, or
the task, Task T4, may be receipt of a sensory stimulation by the subject and the subject maintains the sustained contraction effort at the constant intensity level.

The BBV signal (120) may be used to determine the temporal dynamics of the beta-band sensorimotor rhythm of the brain of the subject.

The BBV signal (120) may be used to determine cortico-muscular coherence (CMC) of the subject.

The proxy signal (122) or BBV signal (120) may be used to determine a status of, monitor a progression of, or be useful in a diagnosis of Parkinson's disease in the subject, wherein an intensity of modulation of the proxy signal (122) or BBV signal (120) strength during the second sub-period is inversely proportional to a severity of the Parkinson's disease.

The proxy signal (122) or BBV signal (120) may be used to determine a status of, monitor a progression of, or monitor recovery from a dysfunction related to cerebrovascular accident, CVA, in the subject, wherein an intensity of modulation of the proxy signal (122) or BBV signal (120) strength during the second sub-period is inversely proportional to a severity of the CVA.

The proxy signal (122) or BBV signal (120) may be used to determine a status of, monitor a progression of, or monitor response to treatment of schizophrenia, in the subject, wherein an intensity of modulation of the proxy signal (122) or BBV signal (120) strength during the second sub-period is inversely proportional to a severity of schizophrenia.

The proxy signal (122) or BBV signal (120) may be used to determine a status of, monitor a progression of, or monitor response to intervention of Autism Spectrum Disorder, ASD, or Developmental coordination disorder, DCD, in the subject, wherein a ratio of proxy signal (122) or BBV signal (120) strength during the second sub-period when the task is T3 or T4 to the proxy signal (122) or BBV signal (120) strength during the second sub-period when the task is T1 or T2, is inversely proportional to the severity of ASD, or DCD.

Provided herein is a measurement device comprising the vibration sensing unit, the measurement device configured to carry out a method as described herein.

Provided herein is a measurement device for determining a proxy for cerebral cortex-detected beta sensorimotor cortical rhythms of the subject, proxy signal (122), wherein the proxy signal (122) is correlated to the cerebral cortex-detected beta sensorimotor cortical rhythms of the subject, the measurement device comprising:
a vibration sensing unit (200); and
a processor, wherein the processor is configured to:
receive a dataset containing measurements from the vibration sensing unit of measured mechanical vibrations of a body part (300), the measurements acquired during a measurement period and during the sustained contraction effort;
extract from the dataset a beta-band vibration signal, BBV signal (120), which is a signal representative of a temporal envelope of the measured mechanical vibrations filtered within a beta frequency band; and
determine from the BBV signal (120), the proxy signal.

With respect to the measurement device:
the measurement period may contain a first sub-period and a second sub-period of measurement;
during the first sub-period of measurement, the sustained contraction effort may be at a constant intensity level; and
during the second sub-period of measurement, the subject may be engaged in a task.

With respect to the measurement device:
the task, Task T1, may be a physical movement performed by the subject and the subject maintains the sustained contraction effort at the constant intensity level; or
the task, Task T2, may be a change in intensity of the sustained contraction effort by the subject from the constant, first, intensity level of the first sub-period to a second intensity level different from the first intensity level; or
the task, Task T3, may be visual observing of or an imagining of a physical movement by the subject and the subject maintains the sustained contraction effort at the constant intensity level; or
the task, Task T4, may be a receipt of a sensory stimulation by the subject and the subject maintains the sustained contraction effort at the constant intensity level.

With respect to the measurement device, the processing unit may be further configured to determine a status of, monitor a progression of, or is useful in a diagnosis of Parkinson's disease in the subject from the proxy signal (122), wherein an intensity of modulation of the proxy signal (122) strength during the second sub-period is inversely proportional to a severity of the Parkinson's disease.

With respect to the measurement device, the processing unit may be further configured to determine a status of, monitor a progression of, or monitor recovery from a dysfunction related to cerebrovascular accident, CVA, in the subject from the proxy signal (122), wherein an intensity of modulation of the proxy signal (122) strength during the second sub-period is inversely proportional to a severity of the CVA.

With respect to the measurement device, the processing unit may be further configured to determine a status of, monitor a progression of, or monitor response to treatment of schizophrenia, in the subject from the proxy signal (122), wherein an intensity of modulation of the proxy signal (122) strength during the second sub-period is inversely proportional to a severity of schizophrenia.

With respect to the measurement device, the processing unit may be further configured to determine a status of, monitor a progression of, or monitor response to intervention for Autism Spectrum Disorder, ASD, or Developmental coordination disorder, DCD, in the subject from the proxy signal (122), wherein a ratio of the proxy signal (122) strength during the second sub-period when the task is T3 or T4 to the proxy signal (122) strength during the second sub-period when the task is T1 or T2, is inversely proportional to the severity of ASD, or DCD.

With respect to the measurement device, the vibration sensing unit (200) may comprise a mechanical force sensor (210) and/or a movement sensor (220), and the BBV signal (120) is extracted from an output of the mechanical force sensor (210) and/or the movement sensor (220).

With respect to the measurement device, the vibration sensing unit (200) may comprise a mechanical force sensor (210), and the measurement device further comprises:
a housing having force-receiving surface configured for receiving the sustained contraction effort by the subject, wherein a part of the force receiving surface is disposed with a sensing body for transmission of the contraction force to the mechanical force sensor;
wherein the processor is configured to extract the BBV signal (120) from an output of the mechanical force sensor (210).

With respect to the measurement device:
the vibration sensing unit (200) may comprise a movement sensor (220);
the measurement device may further comprise a fixation element for attachment of the vibration sensing unit to a part of a limb of the subject for measurement of the one or more primary muscles during the sustained contraction effort of a subject; and
the processor may be configured to extract the BBV signal (120) from an output of the movement sensor (220).

The measurement device may further comprise a display (e.g. display, one or more indicator lights), wherein the processor is configured to send control signals to the display indicating a compliance by the subject with and/or a deviation by the subject from a pre-set sustained contraction effort intensity, using the extracted BBV signal (120) or proxy signal (122).

Provided herein is a computer-implemented method for determining a proxy for cerebral cortex-detected beta sensorimotor cortical rhythms of the subject, proxy signal (122), wherein the proxy signal is correlated to the cerebral cortex-detected beta sensorimotor cortical rhythms of the subject, the method comprising:
receiving a dataset containing measurements from a vibration sensing unit (200) configured to measure mechanical vibrations of a body part (300), the measurements acquired from the subject during a measurement period and during a sustained contraction effort;
extracting from the dataset a beta-band vibration signal, BBV signal (120), which is a signal representative of temporal envelope of the measured mechanical vibrations filtered within a beta frequency band;
wherein the proxy signal (122) is the BBV signal (120), or is derived from the BBV signal (120).

The method may be an offline method or an online method.

The method may further comprise the subject matter of other steps described herein

The vibration sensing unit may comprise a mechanical force sensor, the measurement device further comprising:
a housing having force-receiving surface configured for receiving the sustained contraction effort by the subject, wherein a part of the force receiving surface is disposed with a sensing body for transmission of the contraction force to the mechanical force sensor;
a display (e.g. display, one or more indicator lights);
a processor;
wherein the processor is configured to send control signals to the display indicating a compliance by the subject with and/or a deviation by the subject from a pre-set sustained contraction effort intensity, using signals from the mechanical force sensor.

The BBV signal may be extracted from (an output of) the mechanical force sensor.

The vibration sensing unit may further comprise a movement sensor, the measurement device further comprising:
a fixation element for attachment of the vibration sensing unit to a part of a limb of the subject for measurement of the one or more primary muscles during the sustained contraction effort of a subject;
a processor;
wherein the processor is configured to extract the BBV signal from (an output of) the movement sensor.

Provided herein is a computing device or system configured for performing the method as described herein.

Provided herein is a computer program or computer program product having instructions which when executed by a computing device or system cause the computing device or system to perform the method as described herein.

### FIGURE LEGENDS

FIG. 1 is an illustration of a hand of a subject fitted with the vibration sensing unit.

FIG. 2 is an illustration of a BBV signal or proxy signal evolution over time during the 1^{st} and 2^{nd} sub-periods.

FIG. 3 is an illustration of a BBV signal or proxy signal during the 1^{st} and 2^{nd} sub-periods in a subject having Parkinson's disease, wherein the 2^{nd} sub-period task is T1 or T2, at different times during a measurement programme (chronological order a, b, c).

FIG. 4 is an illustration of a BBV signal or proxy signal during the 1^{st} and 2^{nd} sub-periods in a subject who had a Cerebrovascular accident (CVA), wherein the 2^{nd} sub-period task is T1 or T2, at different times during a measurement programme (chronological order a, b, c).

FIG. 5 is an illustration of a BBV signal or proxy signal during the 1^{st} and 2^{nd} sub-periods in a subject suffering from Schizophrenia, wherein the 2^{nd} sub-period task is T1 or T2, at different times during a measurement programme (chronological order a, b, c) FIG. 6A is an illustration of a BBV signal or proxy signal during the 1^{st} and 2^{nd} sub-periods in a subject suffering from Autism spectrum disorder (ASD), wherein the 2^{nd} sub-period task is T1 or T2 (dashed line) and T3 or T4 (solid line).

FIG. 6B is an illustration of BBV signals or proxy signals measured some time after the BBV signals or proxy signals in FIG. 6A in a measurement programme, wherein the 2^{nd} sub-period task is T1 or T2 (dashed line) and T3 or T4 (dot-dashed line).

FIG. 7A is an illustration of BBV signals or proxy signals during the 1^{st} and 2^{nd} sub-periods in a subject suffering from Developmental co-ordination disorder (DCD), wherein the 2^{nd} sub-period task is T1 or T2 (dashed line) and T3 or T4 (solid line).

FIG. 7B is an illustration of BBV signals or proxy signals measured some time after the BBV signals or proxy signals in FIG. 7A in a measurement programme, wherein the 2^{nd} sub-period task is T1 or T2 (dashed line) and T3 or T4 (dot-dashed line).

FIG. 8 shows experimental data. Panel A shows amplitude (grey intensity) of EEG sensorimotor cortical rhythms in the brain of the subject in the 1^{st} and 2^{nd} sub-periods where task was T1. Panel B shows amplitude (grey intensity) of measurements made using the vibration sensing unit (force transducer) in the 1^{st} and 2^{nd} sub-periods, and at frequencies within the range 0 to 90 Hz. Panel C shows a temporal envelope derived from Panel B covering the 1^{st} and 2^{nd} sub-periods from the vibration signal, wherein the output vibration signal has been filtered to contain mechanical vibration components in the beta frequency band 17 to 23 Hz.

FIG. 9 panel A shows an output vibration signal from the vibration sensing unit during a 1^{st} and 2^{nd} sub-period (all frequencies); panel B shows a profile of a band pass filter to be applied to the output vibration signal of panel A; panel C shows the resulting filtered vibration signal and its upper temporal envelope; panel D shows the BBV signal or proxy signal, which is the average across multiple repetitions of the task of the temporal envelope of panel C.

### DETAILED DESCRIPTION

Before the present method and device of the invention are described, it is to be understood that this invention is not limited to particular systems and methods or combinations described, since such methods and devices and combinations may, of course, vary. It is also to be understood that the terminology used herein is not intended to be limiting.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

It is to be understood that other embodiments may be utilised and structural or logical changes may be made without departing from the scope of the present invention. The following detailed description, therefore, is not to be taken in a limiting sense.

Provided herein is a method for determining a beta band vibrations, BBV, signal of a subject, the method comprising:
obtaining measurements from a vibration sensing unit configured to measure mechanical vibrations caused by a sustained contraction effort of the subject, the measurements (previously) acquired during a measurement period and during the sustained contraction effort,
determining from the measurements a beta-band vibration signal, BBV signal, which is a signal representative of the amplitude (or temporal envelope) of measured mechanical vibrations within a beta frequency band.

The BBV signal is a signal representative of the amplitude of measured mechanical vibrations within a beta frequency band over time during the measurement period. The measured mechanical vibrations are filtered within a beta frequency band. The method is preferably computer implemented. A device may be provided configured to perform the method.

Provided herein is a computer-implemented method for determining a beta band vibrations, BBV, signal of a subject, the method comprising:
receiving a dataset containing measurements from a vibration sensing unit configured to measure mechanical vibrations of a body part of the subject during a sustained contraction effort of the subject, the measurements (previously) acquired during a measurement period and during the sustained contraction effort,
extracting from the dataset a beta-band vibration signal, BBV signal, which is a signal representative of the amplitude (or temporal envelope) of the measured mechanical vibrations within a beta frequency band.

Provided herein is a measurement device for determining a beta band vibrations, BBV, signal (120) of a subject, the measurement device comprising:
vibration sensing unit (200); and
a processor, wherein the processor is configured to:
receive a dataset containing measurements from the vibration sensing unit of measured mechanical vibrations of a body part (300) during a sustained contraction effort of the subject, the measurements acquired during a measurement period and during the sustained contraction effort;
extract from the dataset a beta-band vibration signal, BBV signal (120), which is a signal representative of an amplitude (or temporal envelope) of the measured mechanical vibrations within a beta frequency band over time during the measurement period.

Provided herein is a method or measurement device for determining a proxy for cerebral cortex-detected beta sensorimotor cortical rhythms of the subject (a proxy signal (122)), comprising:
determining a beta band vibrations, BBV, signal (120) of a subject according to a method described herein or using a measurement device described herein;
wherein the proxy signal (122) is the BBV signal (120) or is derived from the BBV signal (120).

Provided herein is a computer-implemented method for determining a proxy for cerebral cortex-detected beta sensorimotor cortical rhythms of the subject, proxy signal (122), wherein the proxy signal is correlated to the cerebral cortex-detected beta sensorimotor cortical rhythms of the subject, the method comprising:
receiving a dataset containing measurements from a vibration sensing unit (200) configured to measure mechanical vibrations of a body part (300), the measurements previously acquired from the subject during a measurement period and during a sustained contraction effort,
extracting from the dataset a beta-band vibration signal, BBV signal (120), which is a signal representative of a temporal envelope of the measured mechanical vibrations filtered within a beta frequency band,
wherein the proxy signal (122) is the BBV signal (120), or is derived from the BBV signal (120).

Provided herein is a measurement device for determining a proxy for cerebral cortex-detected beta sensorimotor cortical rhythms of the subject, proxy signal (122), wherein the proxy signal (122) is correlated to the cerebral cortex-detected beta sensorimotor cortical rhythms of the subject, the measurement device comprising:
vibration sensing unit (200); and
a processor, wherein the processor is configured to:
receive a dataset containing measurements from the vibration sensing unit of measured mechanical vibrations of a body part (300), the measurements acquired during a measurement period and during a sustained contraction effort,
extract from the dataset a beta-band vibration signal, BBV signal (120), which is a signal representative of a temporal envelope of the measured mechanical vibrations filtered within a beta frequency band,
wherein the proxy signal (122) is the BBV signal (120), or is derived from the BBV signal (120).

The proxy for the cerebral cortex-detected beta sensorimotor cortical rhythm signals is a signal(s) (a proxy signal) that is correlated to the cerebral cortex-detected beta sensorimotor cortical rhythms of the subject. The proxy signal is the BBV signal or is derived therefrom. The cerebral cortex refers to the outer layer of gray matter of the brain telencephalon. The cerebral cortex-detected beta sensorimotor cortical rhythm signals are those signals detected using one or more sensors able to detect electrical and/or magnetic activity present within the cerebral cortex of the subject. The one or more sensors typically detect the electrical and/or magnetic activity through the skull of the subject. Examples of cerebral cortex-detected beta sensorimotor cortical rhythm signals include EEG and/or MEG-detected beta sensorimotor cortical rhythm signal detected in the cerebral cortex.

The subject is typically a human subject.

The user of the method is typically a health care specialist (e.g. nurse, physician) or a team of health care specialists.

Where it is mentioned that the measurements have been previously acquired from the subject, it is meant that the dataset has been previously collected from the subject and stored (e.g. on a storage medium, in cloud storage, in temporary memory), and the method is performed using that stored dataset, and not on the body. The data set may be received from data storage (e.g. from a storage medium, from cloud storage, from temporary memory). The method may be an offline method (data set received from data storage). The method may be performed in vitro (not on the human or animal body). The method may be performed in silico (not on the human or animal, within a computer environment).

It is understood that, in an alternative implementation, the method may be online, where the dataset is received from the subject, and the BBV signal extracted while the dataset is being received from the subject.

The inventors have found that during a sustained contraction effort, mechanical vibrations are detectable in the body part which correlate with electroencephalogram (EEG) and/or magnetoencephalography (MEG) sensorimotor measurements in the brain of the subject. In particular, they correlate with EEG-detected and/or MEG-detected beta sensorimotor cortical rhythms in the brain of the subject. Accordingly, the BBV signal (or the proxy signal) is correlated with EEG and/or MEG sensorimotor measurements, in particular with EEG-detected and/or MEG-detected sensorimotor cortical rhythms in the brain of the subject. The sensorimotor cortical rhythms in the brain are primarily in the range 13 to 35 Hz, however, some subjects demonstrate rhythms at frequencies comprised within a range 8 and 40 Hz. The frequency range of sensorimotor cortical rhythms is known as beta range.

The BBV signal is a proxy for cerebral cortex-detected beta sensorimotor cortical rhythm signals of the subject, meaning that the BBV signal or proxy signal is correlated to the cerebral cortex-detected beta sensorimotor cortical rhythms of the subject. Changes (e.g. increase, decrease in amplitude) in the cerebral cortex-detected beta sensorimotor cortical rhythm correlate with changes in the BBV signal or proxy signal.

When the subject is engaged in a task that modulates beta-band sensorimotor cortical oscillations (e.g. momentary increase or decrease in contraction effort; or moving a limb, observing a third person moving a limb, imagining oneself making a movement while maintaining the sustained contraction), the BBV signal is correspondingly modulated.

For more quantitative assessment, the amplitude of power modulations may be rescaled based on an estimation of how efficiently beta-band sensorimotor cortical oscillations are transmitted to peripheral signals. This is described herein as signal variance. This variance shows high inter-individual variability and may be assessed with the coefficient of variation of force or EMG envelope in the beta band during periods where there are no task events.

The invention allows beta-band sensorimotor cortical oscillations—unmodulated or modulated—to be recorded with a minimal setup using a vibration sensing unit. The mechanical vibrations are strongly identifiable against background noise, hence, signal to noise ratio is higher compared with detection and amplification of electrical signals.

A BBV signal or proxy signal can be readily obtained from the vibration sensing unit, which is inexpensive to produce thereby reducing costs for the subject and/or health system. In addition, the measurement does not require expert assistance to collect. Measurements may be taken, for instance, in the home of the subject, or by a general practitioner. It is advantageous, for instance, in remote locations and/or where the subject has limited mobility. The low cost and simplified measurement mean that the health of the subject can be monitored more regularly (e.g. weekly), thereby determining a progression of a disorder.

The method described herein may be used to measure neurological health. It may be used to measure a state of the central nervous system (CNS). It may be used to measure a state of the sensorimotor function.

In particular, the method may be used to measure a primary (lower level) state of the CNS, for instance, to determine beta-band sensorimotor cortical rhythms, or cortico-muscular coherence.

In particular, the method may be used to measure a secondary (higher level) state of the CNS, for instance, dysfunction (Parkinson's disease, cerebrovascular accident, schizophrenia), or disorder (Autism Spectrum Disorder (ASD), Developmental co-ordination disorder (DCD)).

The beta frequency band as used herein is reflective of brainwave frequencies in the sensorimotor brain areas. The beta frequency band is a frequency band having an upper frequency limit and a lower frequency limit, wherein the upper limit, lower limit or both upper limit and lower limit are within a range of sensorimotor cortical rhythms detected in the population (8 and 40 Hz).

Preferably both upper limit and lower limit of the beta frequency band are between 8 and 40 Hz, more preferably between 13 to 35 Hz. The width of the beta frequency band is preferably between 3 Hz and 10 Hz, preferably between 3 Hz and 7 Hz.

Preferably, the beta frequency band upper limit is ≤35 Hz and the lower limit is ≥13 Hz, and the width of the beta frequency band is 5 Hz.

The beta frequency band may not include frequencies <8 Hz and may not include frequencies >40 Hz. The beta frequency band may only include frequencies ≥8 Hz and ≤40 Hz.

The beta-band vibration signal (BBV signal) is an indication of measured mechanical vibrations in the beta band induced in the subject during the sustained contraction effort of the subject. More in particular, it is an amplitude of the beta-band frequency range limited mechanical vibrations. More in particular, it is a temporal amplitude of the beta-band frequency range-limited mechanical vibrations. More specifically, the BBV signal is a temporal envelope of an output vibration signal from the vibration sensing unit that has been limited to (filtered in) the beta band frequency range. Frequencies of the output vibration signal may be limited to the beta band frequency range by application of a filter (e.g. band pass filter, high and/or low pass filter). BBV signal may contain only frequencies of the beta band frequency range. The mechanical vibrations may be measured as force and/or acceleration.

The BBV signal is expressed in a time domain. The time domain, as understood in the art, shows evolution of the mechanical vibration over time during the measurement period. Examples of the BBV signal (120) expressed in the time domain are shown, for instance, in FIGS. 2 to 8C.

The vibration sensing unit generates an output vibration signal indicative of the mechanical vibrations measured during the measurement period. The output vibration signal is transformed into the BBV signal by a series of steps. The steps comprise:
filtering the output vibration signal to contain mechanical vibration components (only) in the beta frequency band,
determining a temporal envelope from the filtered output vibration signal.

The filtering is typically performed by application of a band pass filter. The band pass filter typically has a (pass) band width of 3 Hz to 10 Hz, preferably between 3 Hz and 7 Hz. The band pass filter is typically centred on a frequency, a centre frequency, within the beta frequency band (e.g. centred on a frequency within a range 8 to 40 Hz, preferably 13 to 35 Hz).

The temporal envelope is determined from the filtered output vibration signal. The temporal envelope is a curve outlining one extreme of the filtered vibration signal. As discussed herein, the temporal envelope is the upper (extreme) envelope. However, it is appreciated that the lower (extreme) envelope equally may be used, and the observations mentioned herein apply mutatis mutandis. The temporal envelope may be determined using any suitable method for envelope extraction, including, for instance, a Hilbert transform.

The temporal envelope is determined from the BBV signal. The temporal envelope is the BBV signal. The temporal envelope or BBV signal is limited to mechanical vibration components (only) in the beta frequency band.

FIG. 9 panels A to D schematically illustrates step of filtering and obtaining the temporal envelope. FIG. 9 panel A shows an output vibration signal from the vibration sensing unit during a 1^{st} and 2^{nd} sub-period. Panel B shows a profile of a band pass filter to be applied to the output vibration signal of panel A. Panel C shows the resulting filtered vibration signal and its upper envelope. Panel D shows the BBV signal, which is the average across multiple repetitions of the task of the envelope of panel C.

In practice, a filter bank may be applied to the vibration signal. The filter bank comprises multiple band pass filters, each having a band width of 3 Hz to 10 Hz, preferably 5 Hz. The band width of each band pass filter in the filter bank may or may not be constant. Each band pass filter in the filter bank is centred on a different centre frequency within the beta frequency band. The multiple band pass filters in the filter bank are hence centred on multiple different centre frequencies. The multiple different centre frequencies may be mutually spaced apart by a frequency separation e.g. 1 Hz, 2 Hz, 5 Hz. The frequency separation between pairs of adjacent centre frequencies may be constant or different. The multiple different centre frequencies may be selected so that the band widths of the multiple band pass filters at least partially overlap. Preferably, the multiple band pass filters each have a band width of 5 Hz, and the multiple different centre frequencies are spaced apart by a frequency separation of 1 Hz. The result of applying the filter bank to the vibration signal is multiple filtered vibration signals.

A temporal envelope is extracted from each of the filtered vibration signals. One temporal envelope is selected from the multiple temporal envelopes that is the BBV signal. The selection is made based on the empirical knowledge of how the beta rhythm modulates. Typically, the selected envelope is that showing the maximal reactivity to tasks T1-T2.

Once the temporal envelope is selected, the band width and centre frequency giving rise to that temporal envelope are preferably used for all the measurement periods of the measurement session. The same band width and centre frequency may be used throughout the monitoring programme.

The temporal envelope may be further processed by one or more additional processing steps described below.

The temporal envelope may be normalised (e.g. by division) such that the amplitude during the 1^{st} subperiod has a baseline value of 1. Optionally, there may be further steps of a subtraction of 1 and multiplication by 100% so as to express the BBV signal in percent change from baseline.

The temporal envelope may be rescaled (e.g. by multiplication) using the information of how well beta brain waves are transmitted to peripheral signals. The CMC (cortico-muscular coherence, described below) provides this information, as well as a proxy of it, which is the CV of the BBV signal (CV-BBV) when there is no task (no T1, T2, T3, T4) other than maintaining an isometric contraction. The rescaling of BBV modulation by multiplication by a function of the CV-BBV thus removes effects due to changes in efficiency in transmission of beta-band sensorimotor cortical oscillations to peripheral signals. Changes in transmission efficiency across a monitoring programme can hence be factored out.

The temporal envelope may be divided by its low-frequency trend (the envelope low pass filtered at 0.1 Hz); this may increase robustness by correcting for slow drifts. Such drifts may arise because of changes in the contraction force, changes in mechanical parameters (e.g., a different region of the limb touches the vibration sensing unit), changes in the state of the participant, or other reasons.

The temporal envelope is preferably averaged across multiple repetitions of the task with respect to a transition between the 1^{st} and 2^{nd} sub-periods. Typically, about 50 to 100 repetitions provide a reduction in noise that readily allows observation of a robust task-related modulation in the BBV signal.

The proxy signal is determined from the BBV signal. By determined, it is meant that the proxy signal is the BBV signal, or the proxy signal is derived from the BBV signal. When the proxy signal is the BBV signal, it is meant that the BBV signal is directly used as the proxy signal. When the proxy signal is derived from the BBV signal, it is meant that the BBV signal is transformed into the proxy signal in a manner that the proxy signal represents the information content of the BBV signal, for instance, by scaling in intensity, or by normalising or adding an offset (as guidance, for example, 1 ms to 100 ms, preferably 20 to 40 ms) to account for delay in signal transmission between cerebral cortex and body part. Examples of the proxy signal (122) are shown, for instance, in FIGS. 2 to 8C.

The BBV signal has different characteristics, such as signal strength, and coefficient of variation.

The BBV signal (or proxy signal) is expressed in a time domain. A BBV signal (or proxy signal) is continuously reflective of the cerebral cortex-detected beta sensorimotor cortical rhythms of the subject. A BBV signal (or proxy signal) may have any duration. The duration may depend on actions or tasks performed by the subject. Where there is a 1^{st} and 2^{nd} sub period, the BBV signal (or proxy signal) typically may have a duration of 2 000 to 10 000 ms. A BBV signal (or proxy signal) may be observed for any duration. The duration of observation depends on actions or tasks performed by the subject. Where there is a 1^{st} and 2^{nd} sub period, the duration of observation of the BBV signal (or proxy signal) typically may be 2 000 to 10 000 ms.

The BBV signal (or proxy signal) has a signal strength at any given time during the measurement period, which is related to the BBV signal (or proxy signal). The signal strength is a scalar value. Multiple signal strengths are recorded during the measurement period.

The signal strength may be the amplitude or signal power (square of the signal amplitude). The signal strength may be measured within a moving time window. The time window allows an average signal strength measurement.

A baseline BBV (or proxy) signal strength (SS_{b}) (e.g. amplitude or power) is representative of the BBV (or proxy) signal strength during the sustained contraction without a task (e.g. during a constant contraction effort or during the 1^{st} sub-period (140)). The baseline BBV (or proxy) signal strength may be a scalar value. The baseline BBV (or proxy) signal strength may be determined from one or an average of multiple time points. The baseline BBV (or proxy) signal strength may be used as a normalization factor.

A modulated BBV (or proxy) signal strength (SSₘ) (e.g. amplitude or power) is representative of the BBV (or proxy) signal strength during the 2^{nd} sub-period (160). The modulated BBV (or proxy) signal strength is a scalar value. The modulated BBV (or proxy) signal strength may be determined from one or more time points during the 2^{nd} sub-period. It is understood that the modulated BBV (or proxy) signal strength does not include time points where the signal strength is at baseline during the 2^{nd} sub-period. For instance, it may be determined from one or more time points where the BBV (or proxy) signal initially decreases to a trough (1^{st} event, 162) and/or rises to a peak (2^{nd} event, 164) and/or starts to descend back to baseline without including baseline time points (3^{rd} event, 166) (see FIG. 2). The modulated BBV (or proxy) signal strength may be determined from an average of multiple time points during the 2^{nd} sub-period. Preferably, the modulated BBV (or proxy) signal strength is determined from the BBV (or proxy) signal strength at the trough of the 1^{st} event (162). The chosen point(s) preferably remain the same for determining the modulated BBV (or proxy) signal strength during the measurement period, session or monitoring programme. The modulated BBV (or proxy) signal strength (SSₘ) may be normalised (e.g. by addition or subtraction) by the baseline BBV signal strength, such that the baseline BBV (or proxy) signal strength is equal to a reference value such as zero or one. The modulated BBV (or proxy) signal strength (SSₘ) may alternatively or in addition be rescaled using the coefficient of variation (CV-BBV or CV-proxy); the rescaling factors-out changes in beta wave transmission efficiency by the subject across a monitoring programme.

The BBV (or proxy) signal may be characterized by a coefficient of variation of the BBV (or proxy) signal strength (CV-BBV or CV-proxy). The CV-BBV (or CV-proxy) is a scalar value obtained as the standard deviation of the BBV (or proxy) signal strength (e.g. amplitude) divided by its mean. It is determined while the subject performs a sustained contraction effort at a sustained constant intensity level (e.g. 10%, 30%, 50% of maximum contraction effort) without performing a task. For instance, it may be determined from the baseline (e.g. 1^{st} sub period (140)). The signal variance has a high inter-individual variability.

Contraction effort is preferably isometric. The isometric contraction effort results in a tightening of muscles without a noticeable change in muscle length. Examples include:
application of a mechanical force against an immovable object;
squeezing of an object between finger and thumb;
squeezing of a force sensor between finger and thumb.
The isometric contraction preferably involves (only) one limb.

One or more primary muscles are engaged during the isometric contraction. The one or more primary muscles is a muscle or a group of muscles involved in the sustained contraction effort of a subject. The one or more primary muscles are involved in the isometric contraction effort.

The mechanical vibrations caused by the sustained contraction of the subject are measured in order to determine the BBV signal (or proxy). The mechanical vibrations originate in the one or more primary muscles, and may be transmitted to and detectable in other body parts, via, for instance, bone(s) (e.g. directly connected to the one or more primary muscles) and exterior skin.

The body part may be on the skin, and over the one or more primary muscles. The body part may be on the skin, and over one or more bones directly connected to the one or more primary muscles.

The vibration sensing unit is configured for contact with the skin. The vibration sensing unit may be placed on the skin, and over the one or more primary muscles. The vibration sensing unit may be placed on the skin, and over one or more bones directly connected to the one or more primary muscles.

The measurement period may contain a first sub-period and a second sub-period of measurement. During the first sub-period of measurement, the subject sustains the contraction effort at a first intensity level (e.g. 10% of maximum contraction effort) and without performing any other movement or task (e.g. no movement of limbs, no other isotonic or isometric contraction). During the second sub-period of measurement, the subject performs a Task (T1, T2, T3, T4) while sustaining the contraction effort at the first intensity level (T1, T3, T4) or by changing the contraction effort to a second intensity level (e.g. 15% of maximum contraction effort) different from the first intensity level (T2). This is described in more detail elsewhere herein.

During the second sub-period, there is a modulation over time of the BBV (or proxy) signal compared with the first sub-period.

The second sub-period has an effect of modulating the signal strength (e.g. amplitude) of the BBV (or proxy) signal. This is illustrated, for instance, in FIG. 2, which shows the BBV signal (120) (or proxy signal (122)) evolving over time during the 1^{st} sub period (140) and 2^{nd} sub period (160). Typically, during the first sub-period (140), the BBV signal (120) (or proxy signal (122)) has a steady baseline signal strength, and during second sub-period (160), the BBV signal (120) (or proxy signal (122)) is modulated: the BBV (or proxy) signal strength (e.g. amplitude or power) initially decreases to a trough (1^{st} event, 162), then rises to a peak (2^{nd} event, 164), and then comes back to the initial baseline level (3^{rd} event, 166). The BBV (or proxy) signal strength (e.g. amplitude) of the 1^{st} event is usually considered for measurement of any increase or decrease in modulation.

The sustained contraction effort may be performed at a sustained constant intensity level (e.g. 10%, 30%, 50% of maximum contraction effort) during the whole measurement period and without performing a task during the measurement period.

As mentioned elsewhere herein, from the BBV (or proxy) signal recorded during the constant, non-task, sustained contraction a coefficient of variation for the BBV (or proxy) signal strength (CV-BBV or CV-proxy) may be determined. The CV-BBV (or CV-proxy) is a scalar value obtained as the standard deviation of the BBV (or proxy) signal strength (e.g. amplitude) divided by its mean. The CV-BBV (or CV-proxy) has a high inter-individual variability. The CV-BBV (or CV-proxy) is an indicator of cortico-muscular coherence (CMC) in the subject. As mentioned elsewhere, the CV-BBV (or CV-proxy) may be used to rescale the temporal envelope; changes in beta wave transmission efficiency by the subject across a monitoring programme can hence be factored out.

The measurement period is a duration in time that allows the BBV (or proxy) signal to be determined. As a general guidance, the measurement period may be 1 to 300 s (e.g. for measuring CV-BBV (and/or CV-proxy)), or 1 to 2000 ms (e.g. for measuring BBV signal during 1^{st} and 2^{nd} sub-periods). A measurement session may contain one or multiple measurement periods. The multiple measurement periods may be of the same or different durations. The BBV (or proxy) signal is preferably refined by collecting multiple measurement periods in a measurement session (typically about 50-100 repetitions).

A monitoring programme contains multiple measurement sessions, wherein a majority of the measurement sessions are executed consecutively, each with gap of at least one day between consecutive sessions. A monitoring programme may last several years to allow the state (e.g. deterioration or improvement) of the CNS to be tracked.

The inventors have found that during the sustained contraction effort, the one or more primary muscles mechanically vibrate within the beta frequency band. The mechanical vibrations are small and usually invisible to the eye, but are detectable using the vibration sensing unit. An amplitude of the displacement has been determined to be 0.15 mm on average across participants, with associated force fluctuations of 3 mN.

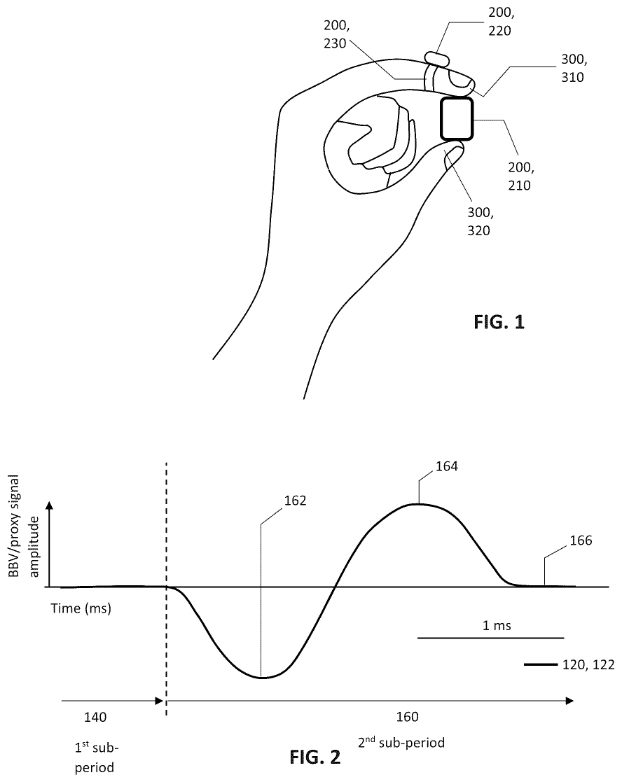

The vibration sensing unit (200) is configured to measure mechanical vibrations of a body part (300). The vibration sensing unit (200) is configured to measure mechanical vibrations of a body part (300) during a sustained contraction effort of the subject. The vibration sensing unit is configured to detect mechanical vibrations with an amplitude of less than 10 to 20 um within the beta frequency band. Typically, the vibration sensing unit comprises a mechanical force sensor and/or a movement sensor. FIG. 1 shows an exemplary vibration sensing unit (200), having a body comprising a mechanical force sensor (210), a movement sensor (220) and a fixation element (230) for attaching the movement sensor (220) to a body part (300) that is a finger (310). Also depicted is a body part (300) that is a thumb (320). The sustained contraction effort of the subject is applied between the finger (310) and thumb (320).

The vibration sensing unit generates an output vibration signal indicative of the mechanical vibrations measured.

The vibration sensing unit may comprise a mechanical force sensor. FIG. 1 shows an exemplary vibration sensing unit (200) comprising the mechanical force sensor (210). The mechanical force sensor is known in the art, and translates an applied mechanical force, such as a compression or tensile force, into an output electrical signal. The mechanical force sensor may receive an applied mechanical force resulting from the sustained contraction effort of the subject (e.g. a squeezing action). The mechanical force sensor is configured to detect variations in the applied force over time, at least in the beta band.

Many different types of mechanical force sensors are known in the art, and typically comprise a pressure transducer that generates an electrical signal as a function of pressure applied. The mechanical force sensor may comprise a load cell, a strain gauge, or a thin film pressure sensor.

Examples of commercially available load cells include those from the AL6B-H family (e.g. AL6B-H-0.6KG-0.4B, AL6B-H-3KG-0.4B). An example of thin-film pressure sensor is the RP-S40-ST.

The vibration sensing unit may comprise a movement sensor. FIG. 1 shows an exemplary vibration sensing unit (200), having the movement sensor (220). The movement sensor may comprise a linear accelerometer. A linear accelerometer is known in the art, and transforms an applied movement into an output electrical signal. The linear accelerometer may measure linear acceleration in one, two or three dimensions.

Additionally or alternatively, the movement sensor may comprise a gyroscope sensor. The gyroscope sensor is known in the art, and transforms applied rotational movement into an output signal. The gyroscope sensor may measure angular velocity in one, two or three dimensions.

Examples of commercially available accelerometers include those from the ADXL family of accelerometers (e.g. ADXL335, ADXL345). Examples of commercially available gyroscopes include those integrated within an inertial motion unit (i.e. combined accelerometer and gyroscope) and manufactured by Witmotion (e.g. WT901C-TTL 9-Axis Vibration Inclinometer).

The movement sensor (220) is configured for attachment to a part of the limb of the subject for measurement of the one or more primary muscles during the sustained contraction effort of a subject. The movement sensor may be configured for attachment over or adjacent to one or more primary muscles. The movement sensor may be configured for attachment to a finger or thumb (e.g. using an elasticated strap, or Velcro strap). FIG. 1 shows an exemplary vibration sensing unit (200), having the movement sensor (220) attached to the finger (310) via a fixation element (230).

As mentioned earlier, the sustained contraction effort is maintained during a measurement period, and the measurement period may contain a first sub-period and a second sub-period of measurement. The BBV signal strength may be measured during the contraction effort in combination with a task (task-based indicator) during the second sub-period.

The BBV (or proxy) signal may be measured during:
a first sub-period of sustained contraction effort at a constant intensity level (e.g. 10% of maximum contraction effort); preferably the subject does not perform any other task during the first sub-period.

a second sub-period of sustained contraction effort during which the subject is engaged in a task (T1 or T2 or T3 or T4).

The BBV (or proxy) signal is measured covering both the first sub-period and second sub-period. Changes in the BBV (or proxy) signal between the first sub-period and second sub-period reflect changes in beta-band sensorimotor cortical oscillations, and hence provide information concerning neurological health, in particular a state of the CNS and/or sensorimotor function.

Modulation in BBV (or proxy) signal strength between the first sub-period and second sub-period is illustrated schematically in FIG. 2. A baseline BBV (or proxy) signal is evident during the 1^{st} sub period (140). Modulation of the BBV (or proxy) signal strength is evident during the 2^{nd} sub period (160). During the 2^{nd} sub period (160), BBV (or proxy) signal strength (e.g. amplitude or power) initially decreases to a trough (1^{st} event, 162), then rises to a peak (2^{nd} event, 164), and then returns back to the baseline (3^{rd} event, 166).

The task may be a physical movement performed by the subject (task T1), a change in intensity level of the contraction effort (task T2), a visual observing of or an imagining of a physical movement by the subject (task T3), or a receipt of sensory stimulation (task T4). Tasks T1, T3 and T4 are performed by the subject during the sustained contraction effort at the same constant intensity level as in the first sub-period; in T2 intensity level of the contraction effort is different from the first sub-period.

The task T1 involves a movement performed by the subject using one or more secondary muscles that are different from the one or more primary muscles. During the second sub-period the subject maintains the sustained contraction effort at the same constant intensity level as in the first sub-period (e.g. 10% of maximum contraction effort). The movement may involve isotonic contractions by the one or more secondary muscles. In one example, the one or more primary muscles may belong to a thumb and finger engaged in a squeezing motion, and the movement using one or more secondary muscles may be a rotation of the wrist joint, and/or rotation of the elbow joint, and/or rotation of the shoulder joint. The movement is preferably transient. The movement is preferably of small amplitude so as not to disturb the sustained contraction effort.

The task T2 involves changing the intensity level by the subject from the (first) intensity level (e.g. 10% of maximum contraction effort) applied during the first sub-period to a different (second) intensity level (e.g. 15% of maximum contraction effort) i.e. the second intensity level is different from the first intensity level. The change to the different (second) intensity level may be transient (e.g. to 15% for few hundreds of ms and then back to 10%).

The task T3 involves a visual observing of or an imagining of a physical movement. During the second sub-period the subject maintains the sustained contraction effort at the same constant intensity level as in the first sub-period (e.g. 10% of maximum contraction effort). The observing may be of another subject (e.g. in the same room), or by visual observing from a display (e.g. computer, table, smart phone) a moving image of a representation of the subject. The imagining may be imagining oneself making a physical movement.

The task T4 is a receipt of a sensory stimulation by the subject. During the second sub-period the subject maintains the sustained contraction effort at the same constant intensity level as in the first sub-period (e.g. 10% of maximum contraction effort). The sensory simulation may be somatosensory, touch, visual, auditory, olfactory, or taste. Examples of somatosensory stimulation, include an air puff, a mechanical vibration or a thermal stimulus. Examples of visual stimuli include light flashes, a checker board pattern reversal, and images of any kind. Examples of auditory stimuli include auditory tones (intermittent), white noise, or vocal sounds.

As mentioned earlier, the BBV (or proxy) signal may be used to measure a primary (lower level) state of the CNS, for instance, to determine beta-band sensorimotor cortical rhythms, or cortico-muscular coherence.

The BBV (or proxy) signal may be used to determine beta-band sensorimotor cortical rhythms of the subject. Changes in the beta-band sensorimotor cortical rhythms of the subject as measured by MEG and/or EEG are reflected in changes in the BBV (or proxy) signal. When the subject enters the 2^{nd} sub-period, beta-band sensorimotor cortical rhythms of the subject change, and the BBV (or proxy) signal is correspondingly modulated. Hence, the BBV (or proxy) signal may be used to determine the temporal dynamics of beta-band sensorimotor rhythms of the brain. Changes (e.g. an increase or decrease) in modulation intensity across a monitoring programme reflect changes in the state of the central nervous system.

The BBV (or proxy) signal may be used to determine cortico-muscular coherence of the subject. CMC refers to a synchrony in the activity of sensorimotor cortical areas and muscle in the beta band. The coefficient of variation of BBV (or proxy) signal envelope (CV-BBV or CV-proxy) is close to linearly related to the CMC. Signals corresponding to motor commands are generated in the primary motor cortex of the brain, and in addition, the cortex signals are communicated from the primary motor cortex, along corticospinal pathways to the muscle or group of muscles. Changes in the generation and/or communication of signals in the subject may be determined by measurement of the CMC. Changes (e.g. an increase or decrease) in the CMC across a monitoring programme reflect changes in the state of the central nervous system.

A higher CV-BBV (or CV-proxy) is indicative of more pronounced transmission of beta-band sensorimotor cortical oscillations. A reduction in CV-BBV (or CV-proxy) in a subject across a monitoring programme is indicative of a reduction in generation and/or transmission of beta-band sensorimotor cortical oscillations.

As mentioned earlier, the BBV (or proxy) signal may be used to measure a secondary (higher level) state of the CNS, for instance, dysfunction (Parkinson's disease, cerebrovascular accident, schizophrenia), or disorder (Autism Spectrum Disorder, Developmental Coordination Disorder).

The BBV (or proxy) signal may be used to determine a status of, monitor a progression of, or be useful in a diagnosis of Parkinson's disease, wherein the measurement period comprises the first and the second sub-period. The intensity of modulation of the BBV (or proxy) signal strength (e.g. amplitude) during the second sub-period is inversely proportional to the severity of Parkinson's disease. A reduction in modulation (over a monitoring programme) of the BBV signal strength during the second sub-period is indicative of an increasing severity of Parkinson's disease.

It has been observed that subjects with Parkinson's disease exhibit reduced modulation over time in beta brain wave signal amplitude in response to tasks akin to T1, T2 and T3 (Heinrichs-Graham et al. Cereb Cortex. 2014; 24: 2669-2678 ; Heida et al. Clinical Neurophysiology. 2014; 125: 1819-1825). Importantly, changes in beta brain waves signal amplitude appear causally related to patients' symptoms (Little & Brown. Parkinsonism & Related Disorders. 2014; 20: S44-S48). The reduction in modulation (over a monitoring programme) of the BBV (or proxy) signal strength during the second sub-period may hence be indicative of an increasing severity of Parkinson's disease.

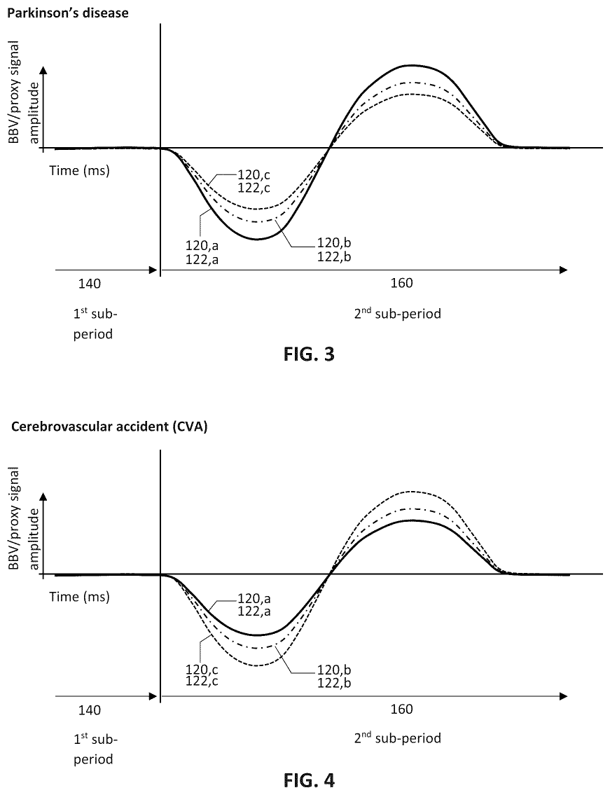

Observing the BBV (or proxy) signal and the modulation of the BBV (or proxy) signal strength caused by the second sub-period, one can expect a smaller (less intense) modulation of the BBV (or proxy) signal to be indicative of a more severe Parkinson's disease. For instance, one can expect a reduction in modulation of the BBV (or proxy) signal strength during the monitoring programme as the condition of Parkinson's disease worsens over time. This is exemplified in FIG. 3, showing the BBV signal (120) (or proxy signal (122)) measured at three different times during a monitoring programme (in chronological order 120,a; 120,b; 120,c/122,a; 122,b; 122,c); the intensity of the modulation of the BBV (or proxy) signal in the second sub-period decreases (120,c/122,c having the lowest modulation) as severity of the Parkinson's disease increases for the subject over time. The reduction in the modulation intensity is greater when the task is T1 or T2 compared with T3 or T4. The intensity of the modulation may be quantified from the modulated BBV (or proxy) signal strength (SSₘ). A reduction in modulated BBV (or proxy) signal strength (SSₘ) across a monitoring programme is indicative of an increase in severity of the Parkinson's disease.

Tremors associated with Parkinson's disease are classically measured using a force sensor and/or accelerometer placed on a limb of the subject. These classic measurements estimate a severity of the disease in terms of amplitude of the tremors (scalar value) in a resting subject. They do not involve sustained contractions, they do not specifically look at the beta band envelope evolving over time, and measurements are not concerned with cerebral cortex-detected beta sensorimotor cortical rhythms of the subject. Classically measured tremors are purely parameters of peripheral physiology, compared with the present method that is concerned with measurements (via a proxy) of the central nervous system, namely, the cerebral cortex and of dysfunctions therein.

The BBV (or proxy) signal may be used to determine a status of, monitor a progression of, monitor recovery from a dysfunction related to cerebrovascular accident (CVA), wherein the measurement period comprises the first and the second sub-period. The intensity of modulation of the BBV (or proxy) signal strength (e.g. amplitude) during the second sub-period is inversely proportional to the severity of a CVA. An increase in modulation (over a monitoring programme) of the BBV (or proxy) signal strength during the second sub-period is indicative of a recovery from a CVA.

It has been observed that subjects demonstrating a recovery from a CVA over time exhibit increased modulation over time in beta brain wave signal amplitude in response to a task in the affected hemisphere, and decreased modulation in the non-affected hemisphere (Espenhahn et al. Brain Commun. 2020, 2: fcaa161; Rossiter et al. J. Neurophysiol. 2014, 112: 2053-2058). The increase in modulation (over a monitoring programme) of the BBV signal strength during the second sub-period is hence indicative of a recovery from a CVA.

Observing the BBV (or proxy) signal and the modulation of the BBV (or proxy) signal strength caused by the second sub-period, one can expect a smaller modulation of the BBV (or proxy) signal in the affected limb to be indicative of a more severe CVA. For instance, one can expect an increase in modulation of the BBV (or proxy) signal strength during the monitoring programme as the dysfunction improves during rehabilitation. This is exemplified in FIG. 4, showing the BBV signal (120) (or proxy signal (122)) measured at three different times during a monitoring programme (in chronological order 120,a; 120,b; 120,c/122,a; 122,b; 122,c), the modulation of the BBV (or proxy) signal in the second sub-period increases (120,c/122,c having the highest modulation) as the subject recovers during rehabilitation. The reduction in the modulation is greater when the task is T1 or T2 compared with T3 or T4.

The intensity of the modulation may be quantified from the modulated BBV (or proxy) signal strength (SSₘ). An increase in modulated BBV (or proxy) signal strength (SSₘ) across a monitoring programme is indicative of a recovery from the CVA.

The BBV (or proxy) signal may be used to determine a status of, monitor a progression of, monitor response to treatment of schizophrenia, where in the second sub-period (160), a task (T1, T2) is performed by the subject. The intensity of modulation of the BBV (or proxy) signal strength (e.g. amplitude) during the second sub-period is inversely proportional to the severity of schizophrenia. An increase in modulation (over a monitoring programme) of the BBV (or proxy) signal strength during the second sub-period is indicative of a recovery from schizophrenia.

It has been observed that subjects with schizophrenia exhibit weaker enhancement in beta brain waves when the task is T1 or T2 (Donati et al., Sci Rep 2021, 11: 15044; Robson et al., NeuroImage: Clinical 2016, 12: 869-878; Hunt et al. Schizophrenia Bulletin 2019, 45: 883-891). An increase in modulation (over a monitoring programme) of the BBV signal strength during the second sub-period when the task is T1 or T2 is hence indicative of a recovery from schizophrenia.

Observing the BBV (or proxy) signal and the modulation of the BBV (or proxy) signal strength caused by the second sub-period, one can expect a smaller modulation of the BBV (or proxy) signal to be indicative of a more severe schizophrenia. For instance, one can expect an increase in BBV (or proxy) signal modulation during the monitoring programme as the dysfunction improves during treatment.

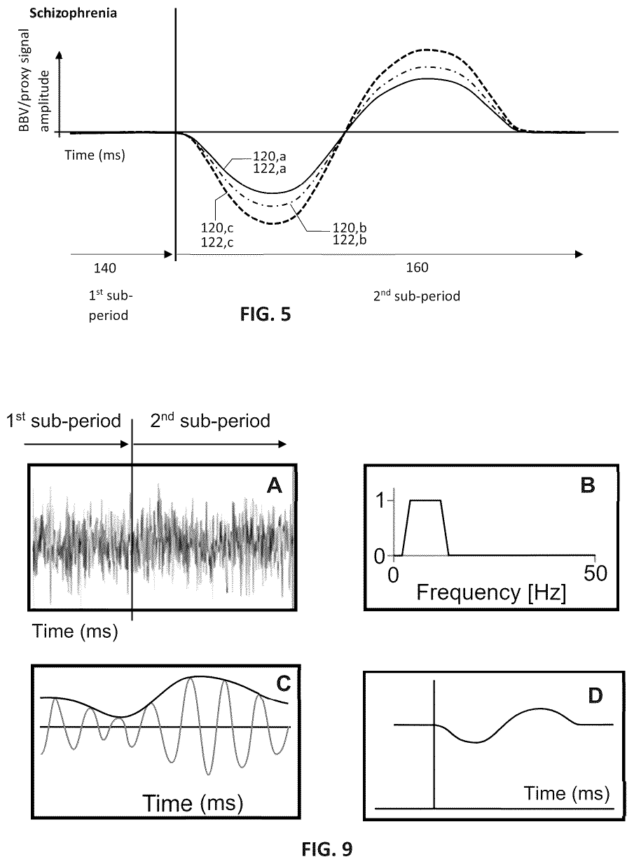

This is exemplified in FIG. 5, showing the BBV signal (120) (or proxy signal (122)) measured at three different times during a monitoring programme (in chronological order 120,a; 120,b; 120,c/122,a; 122,b; 122,c), the intensity of the modulation of the BBV (or proxy) signal in the second sub-period increases (120,c/122,c having the largest modulation) as severity of the schizophrenia decreases for the subject over time. The modulation intensity is greater when the task is T1 or T2 compared with T3 or T4. The intensity of the modulation may be quantified from the modulated BBV (or proxy) signal strength (SSₘ). An increase in modulated BBV (or proxy) signal strength (SSₘ) across a monitoring programme is indicative of a decrease in severity of the schizophrenia.

The BBV (or proxy) signal may be used to determine a status of, monitor a progression of, monitor response to intervention of Autism Spectrum Disorder (ASD), where in the second sub-period (160), a task (T1, T2, T3 or T4) is performed by the subject. The ratio of the modulation in BBV (or proxy) signal strength during the second sub-period when the task is T3 or T4 to the modulation in BBV (or proxy) signal strength during the second sub-period when the task is T1 or T2, is inversely proportional to the severity of the ASD.

It has been observed that subjects with ASD exhibit weaker modulation of the BBV (or proxy) signal strength when the task is T3 or T4 (Bernier et al. Brain Cogn. 2007, 64: 228-37). An increase in modulation (over a monitoring programme) of the BBV (or proxy) signal strength during the second sub-period when the task is T3 or T4 (possibly relative to that when the task is T1 or T2) is hence indicative of a positive evolution of ASD.

Observing the BBV (or proxy) signal and the modulation of the BBV (or proxy) signal strength caused by the second sub-period, the ratio B is calculated:

$$B = ( S {S}_{m} T 3 , T 4 ) / ( {SS}_{m} T 1 , T 2 )$$

Where “SSₘ T3,T4” is the modulated BBV (or proxy) signal strength (SSₘ) during task T3 or T4, and “SSₘ T1, T2” is the modulated BBV (or proxy) signal strength (SSₘ) during task T1or T2.

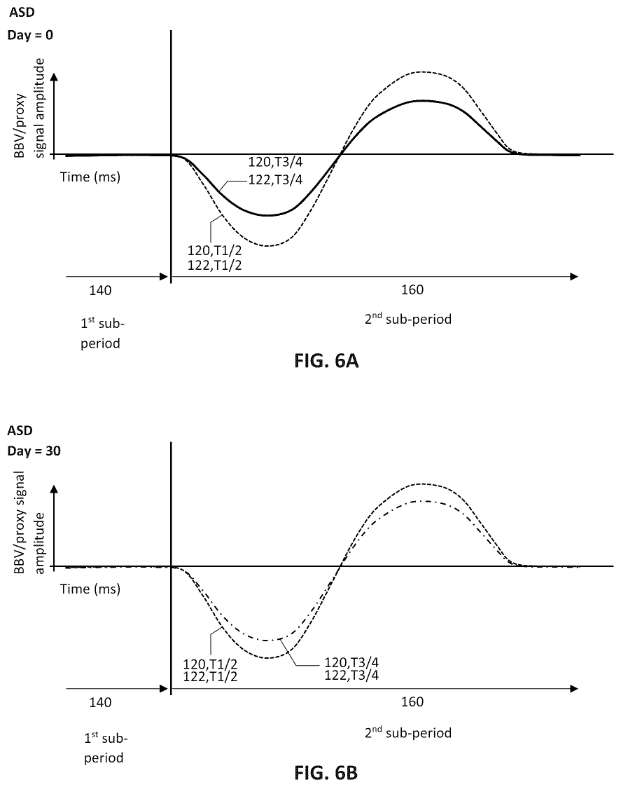

One can expect a smaller ratio B to be indicative of a more severe ASD. For instance, one can expect an increase in ratio B during the monitoring programme as the disorder improves as a result of an intervention. This is exemplified in FIGS. 6A and 6B, showing the BBV signal (120) (or proxy signal (122)) measured at two different times (FIG. 6A earlier than 6B) during a monitoring programme of a subject. In both FIGS. 6A and 6B, the modulated BBV (or proxy) signal strength (SSₘ) (amplitude) during task T1 or T2 (120, T1, T2/122, T1, T2) is greater compared with the modulated BBV (or proxy) signal strength (SSₘ) (amplitude) during task T3 or T4 (120, T3, T4/122, T1, T2). In FIG. 6A the ratio B is smaller than in FIG. 6B, meaning that the disorder was more severe in FIG. 6A than in FIG. 6B.

The BBV (or proxy) signal may be used to determine a status of, monitor a progression of, monitor response to treatment of Developmental coordination disorder (DCD), where in the second sub-period (160), a task (T1, T2, T3 or T4) is performed by the subject. The ratio of the modulation in BBV (or proxy) signal strength during the second sub-period when the task is T3 or T4 to the modulation in BBV (or proxy) signal strength during the second sub-period when the task is T1 or T2, is inversely proportional to the severity of the DCD.

It has been observed that subjects with DCD exhibit weaker modulation of the BBV (or proxy) signal strength when the task is T3 or T4 (Lust et al., Frontiers Hum. Neurosci. 2019, 13: 232). An increase in modulation (over a monitoring programme) of the BBV signal strength during the second sub-period when the task is T3 or T4 (possibly relative to that when the task is T1 or T2) is hence indicative of a positive evolution of DCD.

Observing the BBV (or proxy) signal and the modulation of the BBV (or proxy) signal strength caused by the second sub-period, the ratio C is calculated:

$$C = ( S {S}_{m} T 3 , T 4 ) / ( {SS}_{m} T 1 , T 2 )$$

Where “SSₘ T3,T4” is the modulated BBV (or proxy) signal strength (SSₘ) during task T3 or T4, and “SSₘ T1, T2” is the modulated BBV (or proxy) signal strength (SSₘ) during task T1 or T2.

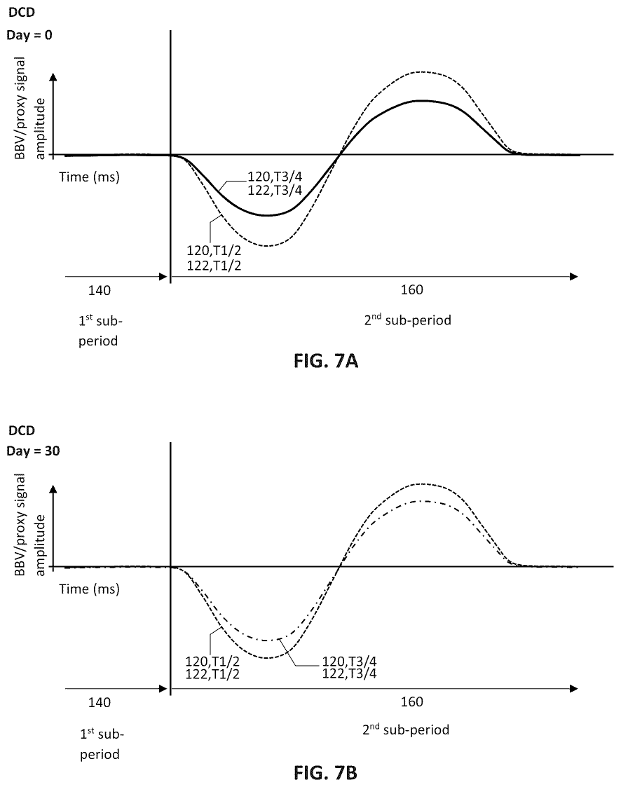

One can expect a smaller ratio C to be indicative of a more severe DCD. For instance, one can expect an increase in ratio C during the monitoring programme as the disorder improves as a result of an intervention. This is exemplified in FIGS. 7A and 7B, showing the BBV signal (120) (or proxy signal (122)) measured at two different times (FIG. 7A earlier than 7B) during a monitoring programme. In both FIGS. 7A and 7B, the modulated BBV (or proxy) signal strength (amplitude) during task T1 or T2 (120, T1 T2/122, T1, T2) is greater compared with the modulated BBV (or proxy) signal strength (amplitude) during task T3 or T4 (120, T3, T4/122, T1, T2). In FIG. 7A the ratio is smaller than in FIG. 7B, meaning that the disorder was more severe FIG. 7A than in FIG. 7B.

The method may comprise receiving EEG data of the subject. The EEG data may comprise beta sensorimotor cortical rhythms in the brain of the subject.

The method may comprise receiving MEG data of the subject. The MEG data may comprise beta sensorimotor cortical rhythms in the brain of the subject.

The EEG data and/or MEG data may be useful as additional information for the user.

Provided here in a measurement device, configured for measurement of a BBV (or proxy) signal according to a method described herein. The measurement device comprises the vibration sensing unit.

The measurement device may comprise:
a housing having force-receiving surface configured for receiving the sustained contraction effort by the subject, wherein a part of the force receiving surface is disposed with a sensing body for transmission of the contraction force to a mechanical force sensor of the vibration sensing unit;
a display (e.g. display, one or more indicator lights);
a processor;
wherein the processor is configured to send control signals to the display indicating a compliance by the subject with and/or a deviation by the subject from a pre-set sustained contraction effort intensity, using signals from the mechanical force sensor.

The BBV signal may be extracted from the mechanical force sensor. The processor may be further configured to extract the BBV signal from the mechanical force sensor. The force receiving surface is preferably configured for holding in the hand of the subject, in particular between the finger(s) and thumb.

The measurement device may comprise:
a movement sensor of the vibration sensing unit,
a fixation element for attachment of the movement sensor to a part of a limb of the subject for measurement of the one or more primary muscles during the sustained contraction effort of a subject;
a processor;
wherein the processor is configured to extract the BBV signal from the movement sensor.

The movement sensor preferably comprises a linear accelerometer and/or a gyroscope sensor. The fixation element (e.g. elasticated strap, Velcro strap) is preferably configured for attachment to a finger or thumb of the subject.

The measurement device may be provided in a smart device (e.g. smart phone) Further provided is a computing device or system configured for performing the method as described herein.

The system comprises circuitry configured to perform the method of the invention. Typically the circuitry comprises a processor and a memory.

Further provided is a computer program or computer program product having instructions which when executed by a computing device or system cause the computing device or system to perform the method as described herein.

Further provided is a computer readable medium having stored thereon instructions which when executed by a computing device or system cause the computing device or system to perform the method as described herein.

The processor may be a part of, or method may be performed using a standard computer system such as an Intel Architecture IA-32 based computer system 2, and implemented as programming instructions of one or more software modules stored on non-volatile (e.g., hard disk or solid-state drive) storage associated with the corresponding computer system. However, it will be apparent that at least some of the steps of any of the described processes could alternatively be implemented, either in part or in its entirety, as one or more dedicated hardware components, such as gate configuration data for one or more field programmable gate arrays (FPGAs), or as application-specific integrated circuits (ASICs), for example. The processor may be a part of, or method may be performed using a single standard computer system and/or a part of system of networked computers.

### EXAMPLE

A subject (n=20) was fitted with a head-fitting cap for measurement of EEG signals at multiple locations on the scalp. The subject was also provided with a vibration sensing unit which could be gripped between the finger and the thumb and contained a force sensor for measuring compression forces applied by the subject. EEG measurements were recorded from the head-fitting cap, and the BBV signal was recorded from the vibration sensing unit.

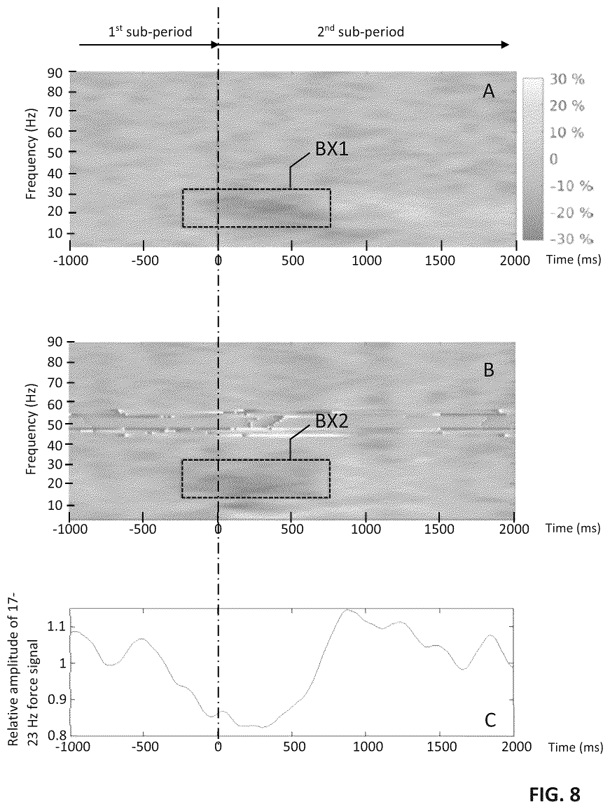

In a first experiment, during each measurement period, the subject was asked to squeeze the vibration sensing unit at a constant intensity for a 1^{st} sub-period and then to move only their elbow during a 2^{nd} sub-period while maintaining the constant intensity. The results are shown in FIG. 8, panels A to C. FIG. 8, panel A shows amplitude (grey intensity) of EEG sensorimotor cortical rhythms in the brain of the subject in the 1^{st} and 2^{nd} sub-periods, and separated into frequency bands within the range 5 to 90 Hz. There is a prominent change in signal amplitude in the beta band indicated with BX1. FIG. 8, panel B shows output vibration signal from a mechanical force sensor of the vibration sensing unit in the 1^{st} and 2^{nd} sub-periods. Grey intensity is force amplitude. The output vibration signal has been separated into frequency bands within the range 5 to 90 Hz. There is a prominent change in signal amplitude in the beta band indicated with BX2 which corresponds with the change in the sensorimotor cortical beta rhythms detected by EEG. FIG. 8, panel C shows a temporal envelope from an output vibration signal from the mechanical force sensor, wherein the output vibration signal has been filtered to contain mechanical vibration components in the beta frequency band 17 to 23 Hz.

1. A computer-implemented method for determining a proxy for cerebral cortex-detected beta sensorimotor cortical rhythms of a subject, a proxy signal, wherein the proxy signal is correlated to the cerebral cortex-detected beta sensorimotor cortical rhythms of the subject, the computer-implemented method comprising:
receiving a dataset containing measurements from a vibration sensing unit configured to measure mechanical vibrations of a body part, the measurements previously acquired from the subject during a measurement period and during a sustained contraction effort;
extracting from the dataset a beta-band vibration signal, a BBV signal, which is a signal representative of a temporal envelope of the measured mechanical vibrations filtered within a beta frequency band;
wherein the proxy signal is the BBV signal, or is derived from the BBV signal.

2. The computer-implemented method according to claim 1, wherein:
the measurement period contains a first sub-period and a second sub-period of measurement;
during the first sub-period of measurement, the sustained contraction effort is at a constant intensity level; and
during the second sub-period of measurement, the subject is engaged in a task.

3. The computer-implemented method according to claim 2, wherein:
the task, Task T1, is a physical movement performed by the subject and the subject maintains the sustained contraction effort at the constant intensity level; or
the task, Task T2, is a change in intensity of the sustained contraction effort by the subject from the constant, first, intensity level of the first sub-period to a second intensity level different from the first intensity level;
the task, Task T3, is visual observing of or an imagining of a physical movement by the subject and the subject maintains the sustained contraction effort at the constant intensity level; or
the task, Task T4, is a receipt of a sensory stimulation by the subject and the subject maintains the sustained contraction effort at the constant intensity level.

4. The computer-implemented method according to claim 2, wherein the proxy signal is used to determine a status of, monitor a progression of, or is useful in a diagnosis of Parkinson's disease in the subject, wherein an intensity of modulation of the proxy or BBV signal strength during the second sub-period is inversely proportional to a severity of the Parkinson's disease.

5. The computer-implemented method according to claim 2, wherein the proxy signal is used to determine a status of, monitor a progression of, or monitor recovery from a dysfunction related to cerebrovascular accident, CVA, in the subject, wherein an intensity of modulation of the proxy signal or BBV signal strength during the second sub-period is inversely proportional to a severity of the CVA.

6. The computer-implemented method according to claim 2, wherein the proxy signal is used to determine a status of, monitor a progression of, or monitor response to treatment of schizophrenia, in the subject, wherein an intensity of modulation of the proxy signal strength during the second sub-period is inversely proportional to a severity of schizophrenia.

7. The computer-implemented method according to claim 3, wherein the proxy signal is used to determine a status of, monitor a progression of, or monitor response to intervention for Autism Spectrum Disorder, ASD, or Developmental coordination disorder, DCD, in the subject, wherein a ratio of the proxy signal strength during the second sub-period when the task is T3 or T4 to the proxy signal strength during the second sub-period when the task is T1 or T2, is inversely proportional to a severity of ASD, or DCD.

8. A measurement device for determining a proxy for cerebral cortex-detected beta sensorimotor cortical rhythms of a subject, a proxy signal, wherein the proxy signal is correlated to the cerebral cortex-detected beta sensorimotor cortical rhythms of the subject, the measurement device comprising:
a vibration sensing unit; and
a processor, wherein the processor is configured to:
receive a dataset containing measurements from the vibration sensing unit of measured mechanical vibrations of a body part, the measurements acquired during a measurement period and during a sustained contraction effort;
extract from the dataset a beta-band vibration signal, a BBV signal, which is a signal representative of a temporal envelope of the measured mechanical vibrations filtered within a beta frequency band; and
determine from the BBV signal, the proxy signal.

9. The measurement device according to claim 8, wherein:
the measurement period contains a first sub-period and a second sub-period of measurement;
during the first sub-period of measurement, the sustained contraction effort is at a constant intensity level; and
during the second sub-period of measurement, the subject is engaged in a task.

10. The measurement device according to claim 9, wherein:
the task, Task T1, is a physical movement performed by the subject and the subject maintains the sustained contraction effort at the constant intensity level; or
the task, Task T2, is a change in intensity of the sustained contraction effort by the subject from the constant, first, intensity level of the first sub-period to a second intensity level different from the first intensity level; or
the task, Task T3, is visual observing of or an imagining of a physical movement by the subject and the subject maintains the sustained contraction effort at the constant intensity level; or
the task, Task T4, is a receipt of a sensory stimulation by the subject and the subject maintains the sustained contraction effort at the constant intensity level.

11. The measurement device according to claim 9, wherein the processor is further configured to determine a status of, monitor a progression of, or is useful in a diagnosis of Parkinson's disease in the subject from the proxy signal wherein an intensity of modulation of the proxy signal strength during the second sub-period is inversely proportional to a severity of the Parkinson's disease.

12. The measurement device according to claim 9, wherein the processor is further configured to determine a status of, monitor a progression of, or monitor recovery from a dysfunction related to cerebrovascular accident, CVA, in the subject from the proxy signal wherein an intensity of modulation of the proxy signal strength during the second sub-period is inversely proportional to a severity of the CVA.

13. The measurement device according to claim 9, wherein the processor is further configured to determine a status of, monitor a progression of, or monitor response to treatment of schizophrenia, in the subject from the proxy signal, wherein an intensity of modulation of the proxy signal strength during the second sub-period is inversely proportional to a severity of schizophrenia.

14. The measurement device according to claim 10, wherein processor is further configured to determine a status of, monitor a progression of, or monitor response to intervention for Autism Spectrum Disorder, ASD, or Developmental coordination disorder, DCD, in the subject from the proxy signal, wherein a ratio of the proxy signal strength during the second sub-period when the task is T3 or T4 to the proxy signal strength during the second sub-period when the task is T1 or T2, is inversely proportional to a severity of ASD, or DCD.

15. The measurement device according to claim 8, wherein the vibration sensing unit comprises a mechanical force sensor and/or a movement sensor, and the BBV signal is extracted from an output of the mechanical force sensor and/or the movement sensor.

16. The measurement device according to claim 8, wherein the vibration sensing unit comprises a mechanical force sensor, and the measurement device further comprises:
a housing having a force-receiving surface configured for receiving the sustained contraction effort by the subject, wherein a part of the force-receiving surface is disposed with a sensing body for transmission of a contraction force to the mechanical force sensor;
wherein the processor is configured to extract the BBV signal from an output of the mechanical force sensor

17. The measurement device according to claim 8, wherein
the vibration sensing unit comprises a movement sensor;
the measurement device further comprises a fixation element for attachment of the vibration sensing unit to a part of a limb of the subject for measurement of one or more primary muscles during the sustained contraction effort of a subject; and
the processor is configured to extract the BBV signal from an output of the movement sensor.

18. The measurement device according to claim 8, further comprising a display (e.g. display, one or more indicator lights), wherein the processor is configured to send control signals to the display indicating a compliance by the subject with and/or a deviation by the subject from a pre-set sustained contraction effort intensity, using the extracted BBV signal or proxy signal.

19. A computer-implemented method for determining a proxy for cerebral cortex-detected beta sensorimotor cortical rhythms of a subject, a proxy signal wherein the proxy signal is correlated to the cerebral cortex-detected beta sensorimotor cortical rhythms of the subject, the method comprising:
receiving a dataset containing measurements from a vibration sensing unit configured to measure mechanical vibrations of a body part, the measurements acquired from the subject during a measurement period and during a sustained contraction effort;
extracting from the dataset a beta-band vibration signal, a BBV signal, which is a signal representative of temporal envelope of the measured mechanical vibrations filtered within a beta frequency band;
wherein the proxy signal is the BBV signal, or is derived from the BBV signal

20. The computer-implemented method according to claim 19, wherein the computer-implemented method is an offline method or an online method.

21. (canceled)
